(19) United States
(12) Patent Application Publication
(10) Pub. No.: US 2024/0206887 A1
(43) Pub. Date: Jun. 27, 2024

(54) Intraosseous Modular Power

(71) Applicant: Bard Access Systems, Inc., Salt Lake City, UT (US)

(72) Inventors: Daniel Pett, Sandy, UT (US); Jason Edie, Sandy, UT (US)

(21) Appl. No.: 18/599,077

(22) Filed: Mar. 7, 2024

Related U.S. Application Data
(63) Continuation of application No. 17/667,291, filed on Feb. 8, 2022, now Pat. No. 11,925,361.
(60) Provisional application No. 63/147,119, filed on Feb. 8, 2021.

(51) Int. Cl. A61B 17/16; A61B 10/02; A61B 17/34; H02J 7/00

(52) U.S. Cl. CPC A61B 17/1628; A61B 17/1637; A61B 17/3472; H02J 7/0042; A61B 2010/0258; H02J 2207/20

(57) ABSTRACT

An intraosseous access system, including a needle configured to drill into bone via rotation of the needle, and a driver. The driver can be configured to impart rotational power to the needle. The driver can include a power converter and a first power source connected to the power converter. The system can further include a second power source external to the driver and selectively connectable to the driver. A method of drilling through a bone includes providing the intraosseous access system, applying rotational power to the needle, and placing the needle in contact with the bone. The method can further include coupling the second power source to the power converter so that power from the second power source is combined with power from the first energy source.

### PRIORITY

This application is a continuation of U.S. patent application Ser. No. 17/667,291, filed Feb. 8, 2022, now U.S. Pat. No. 11,925,361, which claims the benefit of priority to U.S. Provisional Application No. 63/147,119, filed Feb. 8, 2021, each of which is incorporated by reference in its entirety into this application.

### BACKGROUND

Many devices, systems, and methods have been developed to for accessing an interior of a bone of a patient, including for such purposes as intraosseous access, drawbacks that can be resolved, remedied, ameliorated, or avoided by certain embodiments described herein. Intraosseous (“IO”) access systems are often required to access the medullary cavity of bones of different sizes. In some instances, battery operated IO access systems may run out of power when drilling larger bones. Replacing a battery pack during the drilling process may require disengaging the IO access systems from the patient causing a significant procedural interruption and placing the patient at a greater risk. Having a second modular power source at the ready that may be coupled to the IO access system to extend the duration of the drilling process while the device is engaged with the patient may be logistically advantageous for the clinician and may reduce risk to the patient.

### SUMMARY

Disclosed herein is an intraosseous (IO) access system, including an access assembly having a needle configured to drill into bone via rotation of the needle and a driver. The driver includes a housing, a power converter configured to impart rotational power to the needle, a first power source coupled to the power converter, and a second power source selectively coupleable to the driver, where the second power source is configured to be disposed at least partially external to the housing.

The second power source may be a self-contained power source. The power converter may include a gear assembly configured to convert an input rotational speed of an input shaft to an output rotational speed of an output shaft that is different from the input rotational speed.

The driver may be configured to operate with power supplied individually by either the first power source or the second power source. The power converter may be configured for simultaneous coupling with the first power source and the second power source. Simultaneous coupling of the first power source and the second power source with the power converter may provide for extended operational duration of the system. In some embodiments, simultaneous coupling of the first power source and the second power source with the power converter provides for enhanced torque of the needle.

In some embodiments, the first power source is disposed within the housing and the first power source may be replaceable during use. At least one of the first power source or the second power source may be renewable.

In some embodiments, the driver includes a trigger configured to regulate the rotational speed of the needle.

In some embodiments, the first power source is an electrical power source, and the power converter includes an electric motor.

The second power source may also be an electrical power source, and simultaneous coupling of the first power source and the second power source with the power converter may provide for enhanced electrical current supplied to the motor. In some embodiments, the second power source is coupleable to the driver via a wired connection.

In some embodiments, the first power source is at least partially renewable via the second power source.

In some embodiments, the second power source is a mechanical power source.

Disclosed herein also is a method of drilling through a bone. The method includes providing an intraosseous (IO) access system. The IO access system includes an access assembly including a needle configured to drill into bone via rotation of the needle and a driver. The driver includes a housing, power converter configured to impart rotational power to the needle, a first power source coupled to the power converter, the first power source disposed at least partially within the housing, and a self-contained second power source coupleable to the power converter, the second power source disposed external to the housing. The method further includes applying rotational power to the needle and placing the needle in contact with the bone. The method may further include accessing a medullary cavity of the bone.

The method may further include determining that the first power source contains insufficient energy to drill through a cortex of the bone.

The method may further include coupling the second power source to the power converter.

The method may further include adding power supplied by the second power source to power supplied by the first power source.

The first power source may be an electrical power source. In some embodiments, the second power source is an electrical power source, and in alternative embodiments, the second power source is not an electrical power source.

These and other features of the concepts provided herein will become more apparent to those of skill in the art in view of the accompanying drawings and following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates an exploded view of an embodiment of an intraosseous access medical device system, wherein an access assembly subset of the system is depicted slightly enlarged and in elevation, and an automated driver component is depicted in perspective, in accordance with some embodiments herein.

FIG. 2A illustrates a perspective view of an embodiment of an intraosseous driver having an electrical energy source, in accordance with some embodiments.

FIG. 2B illustrates a block diagram depicting various elements of the driver of FIG. 2A, in accordance with some embodiments.

FIG. 3 illustrates a perspective view of another embodiment of an intraosseous driver having a mechanical energy source, in accordance with some embodiments.

FIG. 4 illustrates a perspective view of another embodiment of an intraosseous driver having a pneumatic energy source, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a needle disclosed herein includes a portion of the needle intended to be near a clinician when the needle is used on a patient. Likewise, a “proximal length” of, for example, the needle includes a length of the needle intended to be near the clinician when the needle is used on the patient. A “proximal end” of, for example, the needle includes an end of the needle intended to be near the clinician when the needle is used on the patient. The proximal portion, the proximal-end portion, or the proximal length of the needle can include the proximal end of the needle; however, the proximal portion, the proximal-end portion, or the proximal length of the needle need not include the proximal end of the needle. That is, unless context suggests otherwise, the proximal portion, the proximal-end portion, or the proximal length of the needle is not a terminal portion or terminal length of the needle.

With respect to “distal,” a “distal portion” or a “distal-end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal-end portion, or the distal length of the needle can include the distal end of the needle; however, the distal portion, the distal-end portion, or the distal length of the needle need not include the distal end of the needle. That is, unless context suggests otherwise, the distal portion, the distal-end portion, or the distal length of the needle is not a terminal portion or terminal length of the needle.

In the following description, certain terminology is used to describe aspects of the invention. For example, in certain situations, the term “logic” is representative of hardware, firmware or software that is configured to perform one or more functions. As hardware, logic may include circuitry having data processing or storage functionality. Examples of such circuitry may include, but are not limited or restricted to a hardware processor (e.g., microprocessor with one or more processor cores, a digital signal processor, a programmable gate array, a microcontroller, an application specific integrated circuit “ASIC,” etc.), a semiconductor memory, or combinatorial elements.

Alternatively, logic may be software, such as executable code in the form of an executable application, an Application Programming Interface (API), a subroutine, a function, a procedure, an applet, a servlet, a routine, source code, object code, a shared library/dynamic load library, or one or more instructions. The software may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device. As firmware, the executable code may be stored in persistent storage.

Unless defined otherwise, all technical and scientific terms used herein have the same meaning as commonly understood by those of ordinary skill in the art.

The present disclosure relates generally to bone penetrating devices, systems, and methods. In particular, certain embodiments disclosed herein can be used for drilling through or otherwise being inserted into or penetrating hard, compact bone tissue (cortical bone) to gain access to soft bone tissue (cancellous bone) or bone marrow. For example, certain embodiments are particularly well suited for use in intraosseous access procedures for at least the reasons discussed herein and/or for reasons that are otherwise apparent from the present disclosure.

For purposes of illustration, much of the disclosure herein pertains to creating a conduit or communication passageway to an interior of a bone structure by drilling through or otherwise penetrating hard, compact bone tissue to gain access to bone marrow or cancellous bone. Once access to an inner region of a bone is achieved, any variety of suitable procedures can be performed, such as, for example, infusion, aspiration, or extraction of bone marrow. Numerous situations can benefit from providing access to an interior of a bone in manners such as disclosed herein, such as, for example, when other methods of accessing a vein with an IV needle are difficult or in emergency situations, such as heart attack, burns, drug overdoses, etc., when rapid access to the vasculature of a patient via an interior of a bone may be desired. Other illustrative, non-limiting examples include bone marrow biopsy or bone marrow aspiration. The present disclosure is not, however, limited to these specific applications.

Certain known systems and methods for providing access to bone interior (e.g., bone marrow) rely on a penetrator assembly that includes an outer penetrator and an inner trocar operable by a drill to penetrate the compact bone to gain access to the bone marrow. In order to initially make contact with the hard bone, it is often necessary to penetrate the skin and tissue that covers the bone. The prior methods use a sharp inner trocar in order to poke, puncture, or otherwise advance through the tissue. However, while the sharp tip of the trocar may be suitable for providing a passage through tissue, it can be suboptimal for initiating the cutting action through hard bone. In some instances, the sharp point effectively spins on the surface of the hard bone until the cutting edges of the trocar can become engaged with the hard bone.

Certain embodiments disclosed herein can be advantageous over at least the prior approaches just discussed. For example, in some embodiments, rather than using a sharp-tipped trocar that extends distally beyond cutting surfaces of the outer penetrator, a specialized needle having a distal cutting tip is used. The needle may be coupled with an obturator that does not extend beyond a distal face of the needle and is not involved in cutting or piercing the skin. The needle itself can have both the ability to cut or slice through the skin to reach bone, and can also readily bore through hard bone to the marrow. The obturator can prevent tissue debris from entering the needle lumen during insertion. These and/or other advantages of various disclosed embodiments will be apparent from the discussion that follows.

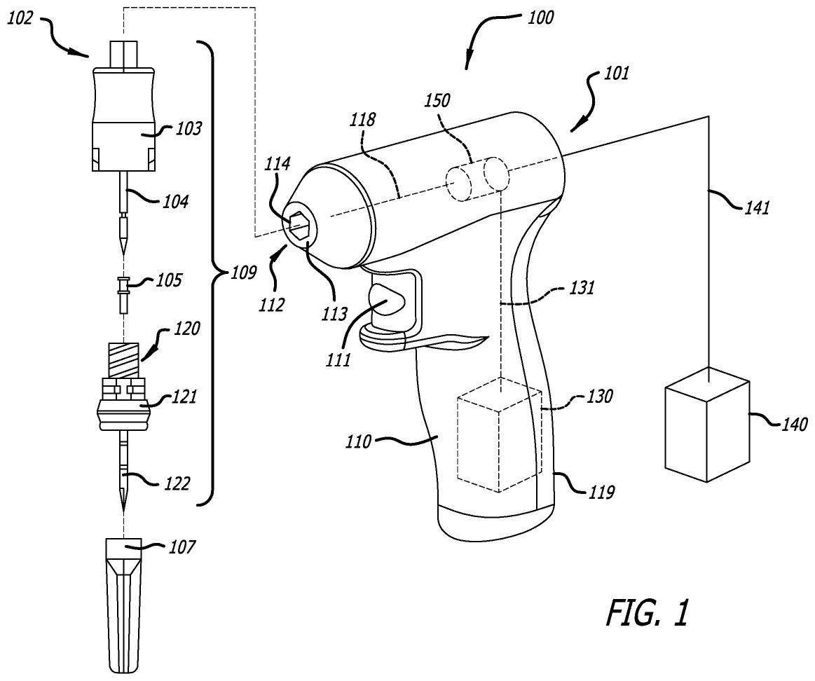

FIG. 1 is an exploded view of an embodiment of an intraosseous (IO) access system 100, with some components thereof shown in elevation and another shown in perspective. The IO access system 100 can be used to penetrate skin and underlying hard bone for intraosseous access, such as, for example to access the marrow of the bone and/or a vasculature of the patient via a pathway through an interior of the bone. The process of drilling through the bone may require power from an energy source.

In various embodiments, the system includes a driver 101 and an access assembly 109. The driver 101 can be used to rotate the access assembly 109 into a bone of a patient. In the illustrated embodiment, the system 100 includes a driver 101 which may be automated. For example, the driver 101 can be a drill that achieves high rotational speeds.

The IO access system 100 can further include an obturator assembly 102, a shield 105, and a needle assembly 120, which may be referred to, collectively, as the access assembly 109. The access assembly 109 may also be referred to as an access system. The obturator assembly 102 is referred to as such herein for convenience. In the illustrated embodiment, the obturator assembly 102 includes an obturator 104. However, in various other embodiments, the obturator 104 may be replaced with a different elongated medical instrument. As used herein, the term “elongated medical instrument” is a broad term used in its ordinary sense that includes, for example, such devices as needles, cannulas, trocars, obturators, stylets, etc. Accordingly, the obturator assembly 102 may be referred to more generally as an elongated medical instrument assembly. In like manner, the obturator 104 may be referred to more generally as an elongated medical instrument.

In the illustrated embodiment, the obturator assembly 102 includes a coupling hub 103 that is attached to the obturator 104 in any suitable manner (e.g., one or more adhesives or overmolding). The coupling hub 103 can be configured to interface with the driver 101, as further discussed below. The coupling hub 103 may alternatively be referred to as an obturator hub 103 or, more generally, as an elongated instrument hub 103.

In the illustrated embodiment, the shield 105 is configured to couple with the obturator 104. The coupling can permit relative longitudinal movement between the obturator 104 and the shield 105, such as sliding, translating, or other movement along an axis of elongation (i.e., axial movement), when the shield 105 is in a first operational mode, and can prevent the same variety of movement when the shield 105 is transitioned to a second operational mode. For example, as further discussed below, the shield 105 may couple with the obturator 104 in a manner that permits longitudinal translation when the obturator 104 maintains the shield 105 in an unlocked state, and when the obturator 104 is moved to a position where it no longer maintains the shield in the unlocked state, the shield 105 may automatically transition to a locked state in which little or no translational movement is permitted between the shield 105 and the obturator 104. Stated otherwise, the shield 105 may be longitudinally locked to a fixed or substantially fixed longitudinal orientation relative to the obturator 104 at which the shield 105 inhibits or prevents inadvertent contact with a distal tip of the obturator, as further discussed below. In various embodiments, the shield 105 may be configured to rotate relative to the obturator 104 about a longitudinal axis of the obturator 104 in one or more of the unlocked or locked states.

With continued reference to FIG. 1, the needle assembly 120 is referred to as such herein for convenience. In the illustrated embodiment, the needle assembly 120 includes a needle 122. However, in various other embodiments, the needle 122 may be replaced with a different instrument, such as, for example, a cannula, a tube, or a sheath, and/or may be referred to by a different name, such as one or more of the foregoing examples. Accordingly, the needle assembly 120 may be referred to more generally as a cannula assembly or as a tube assembly. In like manner, the needle 122 may be referred to more generally as a cannula.

In the illustrated embodiment, the needle assembly 120 includes a needle hub 121 that is attached to the needle 122 in any suitable manner. The needle hub 121 can be configured to couple with the obturator hub 103 and may thereby be coupled with the driver 101, as further discussed below. The needle hub 121 may alternatively be referred to as a cannula hub 121.

In the illustrated embodiment, the shield 105 is configured to couple with the needle hub 121. The coupling can prevent relative axial or longitudinal movement between the needle hub 121 and the shield 105, such as sliding, translating, or the like, when the shield 105 is in the first operational mode, and can permit the shield 105 to decouple from the needle hub 121 when the shield 105 is transitioned to the second operational mode. For example, as further discussed below, the shield 105 may couple with the needle hub 121 so as to be maintained at a substantially fixed longitudinal position relative thereto when the obturator 104 maintains the shield 105 in the unlocked state, and when the obturator 104 is moved to a position where it no longer maintains the shield in the unlocked state, the shield 105 may automatically transition to a locked state relative to the obturator 104, in which state the shield 105 also decouples from the needle hub 121.

As further discussed below, the shield 105 can be coupled with the obturator 104, the obturator 104 can be inserted into the needle 122, and the obturator hub 103 can be coupled to the needle hub 121 to assemble the access assembly 109. In the illustrated embodiment, a cap 107 may be provided to cover at least a distal portion of the needle 122 and the obturator 104 prior to use of the access assembly 109. For example, as further discussed below, in the illustrated embodiment, a proximal end of the cap 107 can be coupled to the obturator hub 103.

With continued reference to FIG. 1, the driver 101 may take any suitable form.

The driver 101 may include a handle 110 that may be gripped by a single hand of a user. The driver 101 may further include an actuator 111 of any suitable variety via which a user may selectively actuate the driver 101 to effect rotation of a coupling interface 112. For example, the actuator 111 may include a switch or other mechanical or electrical element for actuating the driver 101. The actuator 111 may include a button such as a trigger, as shown. In the illustrated embodiment, the coupling interface 112 is formed as a socket 113 that defines a cavity 114. The coupling interface 112 can be configured to couple with the obturator hub 103. In the illustrated embodiment, the socket 113 includes sidewalls that substantially define a hexagonal cavity into which a hexagonal protrusion of the obturator hub 103 can be received.

Other suitable connection interfaces are contemplated.

The driver 101 can include a primary energy (or power) source 130 of any suitable variety that is configured to generate the rotational movement of the coupling interface 112. For example, the primary energy source 130 may provide power in an electrical form (i.e., voltage combined with amperage). In other embodiments, the primary energy source 130 may provide power in a mechanical form (i.e., force combined with velocity or torque combined with rotational speed). In still other embodiments, the primary energy source 130 may provide power in a pneumatic form (i.e., pressure combined with fluid flow).

The driver 101 may include a coupling 131 between the primary energy source 130 and the power converter assembly 150. The coupling 131 is configured to couple the primary energy source 130 to the power converter assembly 150 in any suitable manner consistent with the power from of the primary energy source 130. For example, in the illustrated embodiment, the driver 101 can include an electrical, mechanical, electromechanical, and/or pneumatic coupling 131.

The power converter assembly 150 may be configured to convert a form of power supplied by the primary energy source 130 into rotational power (i.e., torque combined with rotational speed) of the coupling interface 112. For example, the power converter assembly 150 may include a device such as an electrical motor to convert electrical power into rotational power. By way of further example, the power converter assembly 150 may include a gear assembly configured to convert mechanical power supplied by the primary energy source 130 into rotational power of the coupling interface 112. The driver 101 can include a mechanical coupling 118 of any suitable variety to couple the power converter assembly 150 with the coupling interface 112.

Further details and embodiments of the IO access system 100 can be found in WO 2018/075694, WO 2018/165334, WO 2018/165339, U.S. Pat. Nos. 10,893,887, and 10,980,522, each of which is incorporated by reference in its entirety into this application.

With further reference to FIG. 1, the system 100 further includes a secondary energy (or power) source 240. Similar to the primary energy source 130, the secondary energy source 140 may be electrical, mechanical, or pneumatic. In some embodiments, the primary energy source 130 and the secondary energy source 140 may be similar in some respects. For example, in some embodiments, the primary energy source 130 and the secondary energy source 140 may include the same form of energy, e.g., electrical, mechanical, or pneumatic. In other embodiments, the primary energy source 130 and the secondary energy source 140 may include different forms of energy. For example, in some embodiments, the primary energy source 130 may be electrical and the secondary energy source 140 may be mechanical (e.g., rotational).

Similar to the coupling 131, the driver 101 may include a coupling 141 between the secondary energy source 140 and the power converter assembly 150. The coupling 141 is configured to couple the secondary energy source 140 to the power converter assembly 150 in any suitable manner consistent with the power from of the secondary energy source 140. For example, in the illustrated embodiment, the driver 101 can include an electrical, mechanical, electromechanical, and/or pneumatic coupling 141.

In the illustrated embodiment, the primary energy source 130 may be disposed within a housing 119 or at least partially disposed within the housing 119. In other embodiments, primary energy source 130 me be disposed substantially external to the housing 119 or attached to the housing 119. In some embodiments, the primary energy source 130 may form a portion of the driver 101 or the housing 119 such as the handle 110. By way of contrast, the secondary energy source 140 may be disposed external or substantially external to the housing 119. In some embodiments, the secondary energy source 140 may selectively attached to the driver 101 or coupled to the driver 101 via a tether. The secondary energy source 140 may be coupled to the driver 101 such that power from the secondary energy source 140 may be combined with power from the primary energy source 130 to extend an operating duration of the driver 101. The driver 101 may be configured to operate with power provided only by the primary energy source 130. Similarly, in some embodiments, the driver 101 may be configured to operate with power provided only by the secondary energy source 140.

In some instances of use, the primary energy source 130 may contain insufficient energy to complete the drilling process. The cause for the lack of sufficient energy may be a reduced amount (i.e., less than a full capacity) of energy contained within the primary energy source 130 or the full capacity of the primary energy source 130 may insufficient to drill through a bone of a larger size. In either case, it may be necessary to obtain additional energy to complete the drilling process. In some use instances, the clinician may begin a bone drilling process utilizing power from only the primary energy source 130. Upon an indication that the primary energy source 130 may lack sufficient energy to complete the drilling process, the clinician may connect the secondary energy source 140 to the driver 101, thereby extending the operating duration of the driver 101. In other use instances, the clinician may connect the secondary energy source 140 to the driver 101 before starting the bone drilling process.

In some instances, a substantial portion of the energy contained within the primary energy source 130 and/or the secondary energy source 140 may be expended during a bone drilling process. As such, it may be advantageous to replace the energy expended. In some embodiments, the primary energy source 130 and/or the secondary energy source 140 may be renewable, i.e., energy may be added to (i.e., restored to) the primary energy source 130 and/or the secondary energy source 140.

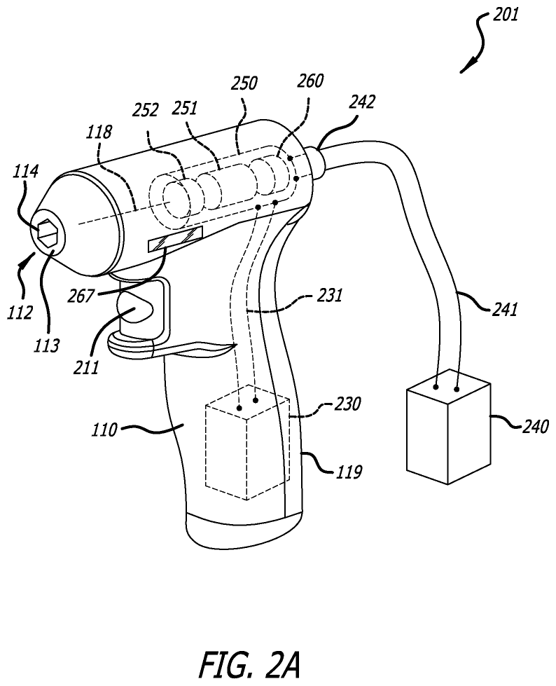

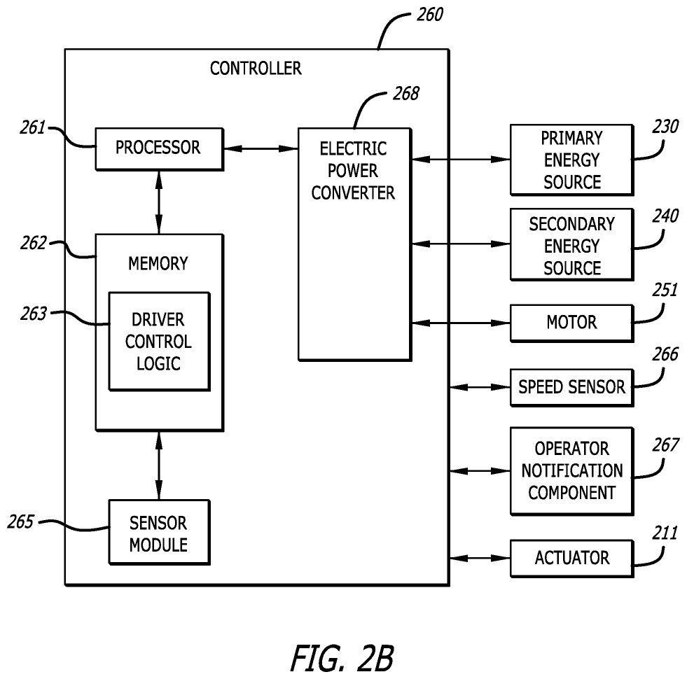

FIG. 2A is a front perspective view of another embodiment of a driver 201 that can, in certain respects, resemble components of the driver 101 described in connection with FIG. 1, and may be included in the system 100. It will be appreciated that all the illustrated embodiments may have analogous features. Accordingly, like features are designated with like reference numerals, with some reference numerals having leading digits incremented to “2.” For instance, the primary energy source is designated as “130” in FIG. 1 and an analogous primary energy source is designated as “230” in FIG. 2A. Relevant disclosure set forth above regarding similarly identified features thus may not be repeated hereafter. Moreover, specific features of the driver 101 and related components shown in FIG. 1 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the driver of FIG. 2A. Any suitable combination of the features, and variations of the same, described with respect to the driver and components illustrated in FIG. 1 can be employed with the driver and components of FIG. 2A, and vice versa. This pattern of disclosure applies equally to further embodiments depicted in subsequent figures and described hereafter.

Referring to FIG. 2A, the driver 210 includes primary and secondary energy sources 230, 240. The primary and secondary energy sources 230, 240 contain energy in the electrical form and provide power to power converter assembly 250 in the electrical form. More specifically, each of the primary and secondary energy sources 230, 240 may include one or more batteries. The primary energy source 230 is coupled to the power converter assembly 250 via electrical conductors 231. Similarly, the secondary energy source 240 is selectively coupleable to the power converter assembly 250 via electrical conductors 241 and a connector set 242. The primary energy source 230 may be disposed within the housing 119 and the secondary energy source 240 may be at least partially disposed external to the housing 119. The connector set 242 may be configured so that the clinician may selectively couple or decouple the secondary energy source 240 with the driver 201.

In the illustrated embodiment, the power converter assembly 250 includes an electric motor 251 configured to convert electrical power into rotational power. In some embodiments, the power converter assembly 250 may include a gear assembly 252 disposed between the electric motor 251 and the coupling 118. In other embodiments, the gear assembly 252 may be omitted.

In some embodiments, the primary energy source 230 and the secondary energy source 240 may be similar in some electrical respects. For example, in some embodiments, the primary energy source 230 and secondary energy source 240 may provide about same voltage. In other embodiments, the primary energy source 230 and secondary energy source 240 may provide different voltages. In some embodiments, the primary energy source 230 and secondary energy source 240 may contain similar amounts of energy when charged. More specifically, the primary energy source 230 and secondary energy source 240 may include the same number of batteries of a similar size. In other embodiments, the primary energy source 230 and secondary energy source 240 may contain different amounts of energy. For example, the secondary energy source 240 may contain about 50 percent, 100 percent, 200 percent or more energy than the primary energy source 230. Alternatively, in some embodiments, the secondary energy source 240 may contain less energy than the primary energy source 230.

In some embodiments, the driver 201 may be connected to an external charger (not shown) to recharge the primary energy source 230. Similarly, the secondary energy source 240 may also be connected to an external charger (not shown) to recharge the secondary energy source 240. In some instances, the driver 201 may be configured such that connecting the secondary energy source 240 to the driver 201 may provide power to the primary energy source 230 to add energy to (i.e., recharge) the primary energy source 230. In some embodiments, the batteries within the one or both of the primary and secondary energy sources 230, 240 may be replaceable by the clinician.

FIG. 2B illustrates a block diagram depicting various elements of the driver 101, in accordance with some embodiments. In some embodiments, the driver 101 may include a controller 260 including a processor 261 and memory 262 which may include a non-transitory computer-readable storage medium having driver control logic 263 stored thereon. The controller 260 may also include an electric power converter 268 and a sensor module 265. Each of the primary energy source 230, the secondary energy source 240, and the motor 251 may be coupled to the electric power converter 268. The sensor module 265 may include an ammeter configured to measure an operating amperage draw of the motor 251, and one or more volt meters configured to measure voltages of the primary and secondary energy sources 230, 240. Also coupled to the controller are the actuator 211 and a motor speed sensor 266. The driver 201 may also include an operator notification component 267 coupled to the controller 260. The operator notification component 267 may be configured to provide visual and/or audible indications to the clinician. In some embodiments, the operator notification component 267 may include a display for rendering indicia pertaining to the operation of the driver 201. In some embodiments, the operator notification component 267 may also include an audio device suitable for providing an audible alert to the clinician during operation of the driver 201. The actuator 211 may be configured to provide a binary signal and/or a variable signal to the controller 260. The motor speed sensor 266 provides a variable signal to the controller 260 indicative of the rotational speed of the motor 251.

The electric power converter 268 may be configured to receive power from the primary energy source 230 and the secondary energy source 240 and supply power to the motor 251. In some embodiments, the electric power converter 268 may include a power supply (e.g. a switching power supply) to convert the voltages of the primary energy source 230 and the secondary energy source 240 into an operating voltage for the motor 251. The electric power converter 268 may be coupled to the processor 261 so that the electric power converter 268 may regulate power supplied to the motor 261 according to the driver control logic 263. In some embodiments, the electric power converter 268 may receive power from the secondary energy source 240 and supply power to the primary energy source 230 to recharge the primary energy source 230.

The driver control logic 263 is configured to receive signal data from one or more sensors and control one or more operating characteristics of the driver 201 when executed by the processor 262. In some embodiments, the driver control logic 263 may collect voltage data from the primary energy source 230, where the voltage measurement may indicate a state of charge for the primary energy source 230. The driver control logic 263 may compare the voltage signal with a defined low voltage limit stored in the memory 262. As a result of the comparison, the driver control logic 263 may provide an indication to the clinician via the operator notification component 267 that the remaining energy contained within the primary energy source 230 is below a low limit. In response, the clinician may couple the secondary energy source 240 to the driver 201. In some instances, the clinician may couple the secondary energy source 240 to the driver 201 before starting the drilling process. In other instances, the clinician may couple the secondary energy source 240 to the driver 201 during the drilling process. In some instances, the driver control logic 263 may provide an indication on the operator notification component 267 of a remaining operational duration for the primary energy source 230.

In some embodiments, the driver control logic 263 may collect voltage data from the secondary energy source 240, wherein the voltage measurement may indicate a state of charge for the secondary energy source 240. The driver control logic 263 may compare the voltage signal with a defined low voltage limit stored in memory 262. As a result of the comparison, the driver control logic 263 may provide an indication to the clinician via the operator notification component 267 that the remaining energy contained within the secondary energy source 240 is below a low limit. In response, the clinician may replace the secondary energy source 240 with another secondary energy source 240. In some instances, the clinician may replace the secondary energy source 240 before starting the drilling process. In other instances, the clinician may replace the secondary energy source 240 during the drilling process. In some instances, the driver control logic 263 may provide an indication on the operator notification component 267 of a remaining operational duration for the secondary energy source 240.

The driver control logic 263 may be configured to regulate the rotational speed of the needle. In some embodiments, empirical studies may have determined an optimal rotational speed range for the needle 122 when drilling through bone and the optimal rotational speed range may be stored in the memory 262. The driver control logic 263 may be configured to receive rotational speed data from the speed sensor 266 and compare the speed data with the optimal rotational speed range stored in memory 262. As a result of the comparison, the driver control logic 263 may adjust a voltage or current supplied to the motor 251 to establish and maintain the rotational speed of the needle 122 to be within the optimal rotational speed range.

In some instances, the clinician may vary the downward force of the needle 122 on the bone while drilling which may in turn vary the torque load on the needle 122. In such instances, the driver control logic 263 may maintain the rotational speed of needle across a varying torque load on the needle 122.

The driver control logic 263 may be configured provide an indication to the clinician that one or more operating parameters of the driver 201 is outside of a defined range. For example, in some embodiments, empirical studies may have determined a high current limit for efficient use of energy from the primary energy source 230 and/or the secondary energy source 240. In some embodiments, the electrical current data may be related to the torque provided by the motor 251. As such, in some embodiments, the driver control logic 263 may receive electrical current data supplied to the motor 251 from the electrical sensor module 265 and compare the current data with a high current limit stored in the memory 262. As a result of the comparison, the driver control logic 263 may provide a visual and/or audible indication to the clinician via the operator notification component 267. In response, the clinician may reduce an applied force to the needle 122 to reduce the torque on the needle 122 and in turn reduce current supplied to the motor 251.

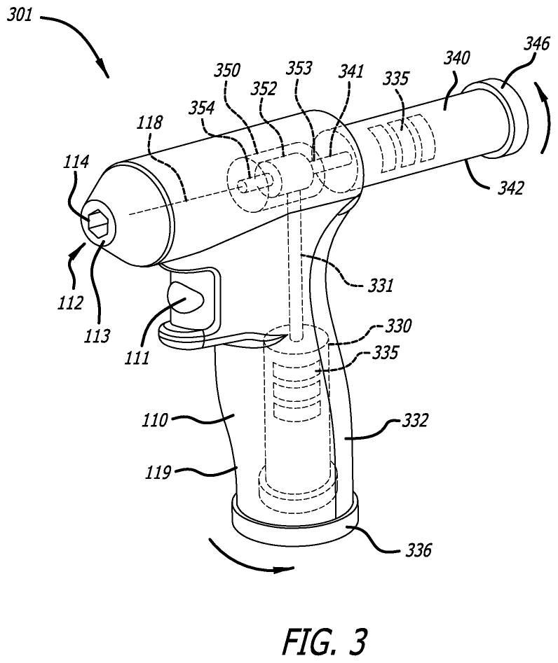

FIG. 3 is a front perspective view of another embodiment of a driver 301 that can, in certain respects, resemble components of the driver 101 described in connection with FIG. 1 and may be included in the system 100. Referring to FIG. 3, the primary and secondary energy sources 330, 340 may provide power to the power converter assembly 350 in a mechanical form. More specifically, the primary and secondary energy sources 330, 340 may provide rotational power to the power converter assembly 350. The primary and secondary energy sources 330, 340 may include one or more torsional springs 335 to provide rotational power (i.e., torque in combination with rotational speed) to the power converter assembly 350. In some embodiments, the torsional springs 335 may be coupled together in a series relationship (i.e., end to end) so that the torque supplied by each of the torsional springs 335 is equal. In some embodiments, the torsional springs 335 may be flat torsional springs. The primary energy source 330 is coupled to the power converter assembly 350 via a rotatable shaft 331. Similarly, the secondary energy source 340 is selectively coupleable to the power converter assembly 350 via a rotatable shaft 341. The primary energy source 330 may be substantially disposed within the housing 119 and the secondary energy source 340 may be at least partially disposed external to the housing 119. The driver 301 may be configured to be operational when either or both of the primary and secondary energy sources 330, 340 is coupled to the power converter assembly 350.

The power converter assembly 350 is configured to convert the rotational power from the primary and secondary energy sources 330, 340 into rotational power of the coupling interface 112. In the illustrated embodiment, the power converter assembly 350 includes a gear assembly 352. The gear assembly 352 may be configured to change the orientation of the rotational power. In other words, the gear assembly 352 may receive rotational power via the rotatable shaft 331 disposed in a first orientation into rotational power of the output shaft 354 disposed in a second orientation, wherein the second orientation is disposed at about 90 degrees with respect to the first orientation. The gear assembly 352 may also be configured to change a rotational speed of the output shaft 354 with respect to a rotational speed of the input shaft 353. In some embodiments, the gear assembly 352 may be configured to increase a rotational speed of the output shaft 354 with respect to a rotational speed of an input shaft 353 by a ratio of about 20 to 1, 50 to 1, 100 to 1, 500 to 1, or more so that the rotational speed of the needle 122 is appropriate for drilling through bone.

In some embodiments, the primary energy source 330 and the secondary energy source 340 may be similar in some respects. For example, in some embodiments, the primary energy source 330 and secondary energy source 340 may interchangeable. In some embodiments, the primary energy source 330 and secondary energy source 340 may contain similar amounts of energy when disposed in a wound-up state. More specifically, the primary energy source 330 and secondary energy source 340 may include the same number of torsional springs 335 of a similar size. In other embodiments, the primary energy source 330 and secondary energy source 340 may contain different amounts of energy. For example, the secondary energy source 340 may contain about 50 percent, 100 percent, 200 percent, or more energy than the primary energy source 330. Alternatively, in some embodiments, the secondary energy source 340 may contain less energy than the primary energy source 330.

The primary energy source 330 may be renewed from a lesser wound-up state to a greater wound-up state. The primary energy source 330 may include a rewind knob 336 coupled to the torsional springs 335. The primary energy source 330 is configured such that turning the rewind knob 336 with respect to a housing 332 of the primary energy source 330 winds up the torsional springs 335 thereby restoring energy to the primary energy source 330. As shown in FIG. 3, the rewind knob 336 may extend beyond the handle 110 making the rewind knob 336 accessible to the clinician when the primary energy source 330 is disposed within the housing 119. As such, the clinician may turn the rewind knob 336 with respect to the handle 110 to wind up the torsional springs 335.

Similarly, the secondary energy source 340 may be renewed from a lesser wound-up state to a greater wound-up state. The secondary energy source 340 may include a rewind knob 346 coupled to the torsional springs 335 so that turning the rewind knob 346 with respect to a housing 342 of the secondary energy source 340 winds up the torsional springs 335 thereby restoring energy to the secondary energy source 340.

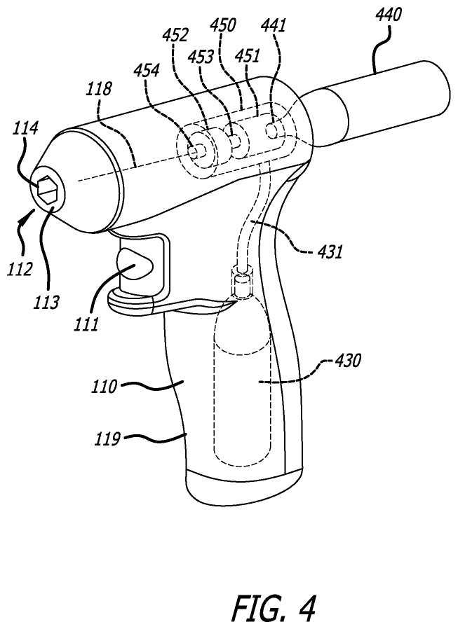

FIG. 4 is a front perspective view of another embodiment of a driver 401 that can, in certain respects, resemble components of the driver 101 described in connection with FIG. 1 and may be included in the system 100. Referring to FIG. 4, the primary and secondary energy sources 430, 440 may provide power to the power converter assembly 450 in a pneumatic form. Each of the primary and secondary energy sources 430, 440 may include a pressurized fluid cartridge (e.g. a CO2 cartridge) to provide pneumatic power (i.e., pressure in combination with fluid flow) to the power converter assembly 450. The primary energy source 430 is coupled to the power converter assembly 450 via a fluid conduit 431. Similarly, the secondary energy source 440 is selectively coupleable to the power converter assembly 450 via a fluid conduit 441. The primary energy source 430 may be substantially disposed within the housing 119 and the secondary energy source 440 may be at least partially disposed external to the housing 119. The driver 401 may be configured to be operational when either or both of the primary and secondary energy sources 430, 440 is coupled to the power converter assembly 450.

In the illustrated embodiment, the power converter assembly 450 includes an air turbine 451 to convert pressurized fluid flow into rotational power of a turbine output shaft 453. In other embodiments, the power converter assembly 450 may include a vane pump, a piston pump, or any other suitable mechanism for converting pressurized fluid flow into rotational power. The power converter assembly 450 may also include gear assembly 452. The gear assembly 452 may be configured to reduce a rotational speed of a gear-assembly output shaft 454 with respect to a rotational speed of the turbine output shaft 453 by a ratio of about 2 to 1, 5 to 1, 10 to 1, 50 to 1, 100 to 1, or more so that the rotational speed of the needle 122 is appropriate for drilling bone.

In some embodiments, the primary energy source 430 and the secondary energy source 440 may be similar in some respects. For example, in some embodiments, the primary energy source 430 and secondary energy source 440 may interchangeable. In some embodiments, the primary energy source 430 and secondary energy source 440 may contain similar amounts of pneumatic energy. More specifically, the primary energy source 430 and secondary energy source 440 may include a cartridge of about the same volume containing a similar mass of fluid. In other embodiments, the primary energy source 430 and secondary energy source 440 may contain different amounts of energy. For example, the secondary energy source 440 may contain about 50 percent, 100 percent, 200 percent or more energy than the primary energy source 430. Alternatively, in some embodiments, the secondary energy source 440 may contain less energy than the primary energy source 430. Either or both of the primary and secondary energy sources 430, 440 may be replaceable by the clinician.

Some embodiments of the system 100 may be configured to include components of any of the drivers 201, 301, and 401. For example, an embodiment of the system 100 may include the driver 201 having the electrical primary energy source 230 combined with the rotational secondary energy source 340 or the pneumatic secondary energy source 440. Embodiments of the system 100 that include other combinations of drivers and secondary energy sources are also contemplated.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

In an exemplary method of the use, the clinician may obtain the system 100.

The clinician may further apply rotational power to the needle by pressing the actuator 111 (trigger). The clinician may then contact the needle 122 with the bone to drill through the bone. At some point before or during the drilling procedure, the clinician may determine that the first energy source 130 contains insufficient energy to complete the drilling procedure. In some embodiments, the system 100 may visually or audibly notify the clinician that the first energy source 130 contains insufficient energy. As a result of the determination, the clinician may couple the second energy source 140 to the driver 101 so that power from the second energy source 140 is combined with power from the first energy source 130. In some embodiments, the first power source 130 is an electrical power source and the second power source 140 is an electrical power source. In other embodiments, the first power source 130 is an electrical power source and the second power source 140 is not an electrical power source.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An intraosseous access system, comprising:
a needle configured to drill into bone via rotation;
a driver configured to impart rotational power to the needle, the driver containing a power converter and a first power source connected to the power converter; and
a second power source external to the driver, the second power source configured for selective connection to the driver to assist in imparting rotational power to the needle during use of the intraosseous access system.

2. The intraosseous access system according to claim 1, wherein the second power source is a self-contained power source.

3. The intraosseous access system according to claim 1, wherein the power converter comprises a gear assembly configured to convert an input rotational speed of an input shaft to an output rotational speed of an output shaft that is different from the input rotational speed.

4. The intraosseous access system according to claim 1, wherein the driver is configured to operate with power supplied either simultaneously by the first power source and the second power source or individually by the first power source or the second power source.

5. The intraosseous access system according to claim 1, wherein the second power source is configured for selective connection directly to the power converter in the driver.

6. The intraosseous access system according to claim 5, wherein combining power from the first power source and the second power source provides for extended operational duration of the intraosseous access system and enhanced torque of the needle.

7. The intraosseous access system according to claim 1, wherein the first power source is replaceable during use.

8. The intraosseous access system according to claim 1, wherein at least one of the first power source or the second power source is renewable.

9. The intraosseous access system according to claim 1, wherein the driver further comprises a trigger configured to regulate the rotational speed of the needle.

10. The intraosseous access system according to claim 1, wherein the first power source is an electrical power source, and wherein the power converter comprises an electric motor.

11. The intraosseous access system according to claim 1, wherein the second power source is an electrical power source.

12. The intraosseous access system according to claim 11, wherein the second power source is configured for connection to the driver via a wired connection.

13. The intraosseous access system according to claim 11, wherein the first power source is at least partially renewable via the second power source.

14. The intraosseous access system according to claim 1, wherein the second power source is a mechanical power source.

15. A method for drilling through a bone, comprising:
providing an intraosseous access system comprising:
a needle configured to drill into the bone via rotation;
a driver configured to impart rotational power to the needle, the driver containing a power converter and a first power source connected to the power converter; and
a second power source external to the driver, the second power source configured for selective connection to the driver to assist in imparting rotational power to the needle during use of the intraosseous access system;
applying rotational power to the needle with the second power source disconnected from the driver; and
placing the needle in contact with the bone.

16. The method according to claim 15, further comprising accessing a medullary cavity of the bone.

17. The method according to claim 15, further comprising:
determining that the first power source contains insufficient energy to drill through a cortex of the bone,
connecting the second power source to the driver, and
drilling through the cortex of the bone.
